(19) United States
(12) Patent Application Publication
(10) Pub. No.: US 2003/0124509 A1
(43) Pub. Date: Jul. 3, 2003

(54) Laminar flow patterning and articles made thereby

(76) Inventors: Paul J.A. Kenis, Champaign, IL (US); Rustem F. Ismagilov, Chicago, IL (US); Shuichi Takayama, Ann Arbor, MI (US); Emanuele Ostuni, Watertown, MA (US); J. Cooper McDonald, Somerville, MA (US); George M. Whitesides, Newton, MA (US)

Correspondence Address: Timothy J. Oyer, Ph.D., Wolf, Greenfield & Sacks, P.C., 600 Atlantic Avenue, Boston, MA, 02210, US

(21) Appl. No.: 10/254,441

(22) Filed: Sep. 25, 2002

Related U.S. Application Data
(63) Continuation of application No. 09/586,241, filed on Jun. 2, 2000, now abandoned.
(60) Provisional application No. 60/137,333, filed on Jun. 3, 1999.
(60) Provisional application No. 60/150,456, filed on Aug. 24, 1999.

(51) Int. Cl. C12M 1/34; B01F 5/06; C12Q 1/00

(52) U.S. Cl. 435/4; 435/287.1; 366/337

(57) ABSTRACT

The present invention provides a variety of techniques for altering a surface using fluid flow. Generally, the invention involves creating adjacent components of a fluid stream exhibiting laminar flow, the first and second components mixing only via diffusion and being free of turbulent mixing. The first and second components are made to flow adjacent first and second portions, respectively, of a surface, and the first and second portions of the surface can be altered, selectively, using the components of the fluid stream. For example, at the first portion of the surface etching, plating, biological deposition, or the like can be carried out, while at the second portion of the surface a different situation can exist. At the second portion the same interaction can be effected as at the first portion, but to a different extent, or a different interaction or no interaction at all can be effected. For example, plating of a metal can occur at the first portion of the surface while nothing can occur at the second portion, or at the second portion of the surface plating to a different extent, or with a different metal, or etching can be made to occur. Alternatively, or in addition, fluid components can be selected where reaction between the fluid components, at the boundary therebetween, can effect a change at a surface against which the boundary flows. Etching, plating, or the like can be carried out in this manner alone, or in addition to selective interactions effected by the first and second components of the fluid adjacent respective first and second portions of the surface. The surface can be tailored to be receptive to any of the described changes, selectively. The invention provides these techniques, and articles produced by these techniques.

### RELATED APPLICATIONS

This application is a continuation of U.S. patent application Ser. No. 09/586,241, filed Jun. 2, 2000, which claims priority to U.S. Provisional Patent Application Serial No. 60/137,333, filed Jun. 3, 1999 and of U.S. Provisional Patent Application Serial No. 60/150,456, filed Aug. 24, 1999.

### GOVERNMENT FUNDING

Research leading to the present invention was funded at least in part by grant no. ECS9729405 from the National Science Foundation. The U.S. Government has certain rights in the invention.

### FIELD OF THE INVENTION

The present invention relates generally to a laminar flow channel system, and more particularly to techniques for carrying out chemical or biochemical interactions at confined regions of surfaces using laminar flow.

### BACKGROUND OF THE INVENTION

A variety of techniques are known for very small-scale fluid flow, for example involving chemical analysis. Typical known techniques involve passing a fluid through a very narrow, confined space and analyzing the content of the fluid. Included among these techniques are chromatography, clinical analysis of physiological fluids, capillary electrophoresis, and the like.

Laminar flow occurs when two or more streams having a certain characteristic (low Reynolds number) are joined into a single stream, also with low Reynolds number, and are made to flow parallel to each other without turbulent mixing. The flow of liquids in capillaries often is laminar. For a discussion of laminar flow and Reynolds number, see Kovacs, G. T. A., Micromachined Transducers Sourcebook (WCB/McGraw-Hill, Boston, 1998); Brody, J. P., Yager, P., Goldstein, R. E. and Austin, R. H., Biotechnology at Low Reynolds Numbers, Biophys. J, 71, 3430-3441 (1996); Vogel, S., Life in Moving Fluids (Princeton University, Princeton, 1994); and Weigl, B. H. and Yager, P., Microfluidic Diffusion-based Separation and Detection, Science 283, 346-347 (1999).

Analytical chemical techniques have utilized laminar flow to control the positioning of fluid streams relative to each other. U.S. Pat. No. 5,716,852 (Yager et al.), describes a chemical sensor including a channel-cell system for detecting the presence and/or measuring the presence of analytes in a sample stream. The system includes a laminar flow channel with two inlets in fluid connection with the laminar flow channel for conducting an indicator stream and a sample stream into the laminar flow channel, respectively. The indicator stream includes an indicator substance to detect the presence of the analyte particles upon contact. The laminar flow channel has a depth sufficiently small to allow laminar flow of the streams and length sufficient to allow particles of the analyte to diffuse into the indicator stream to form a detection area.

U.S. Pat. No. 4,902,629, (Meserol et al.), discusses laminar flow in a description of apparatus for facilitating reaction between an analyte in a sample and a test reagent system. At least one of the sample and test reagent system is a liquid, and is placed in a reservoir, the other being placed in a capillary dimensioned for entry into the reservoir. Entry of the capillary into the reservoir draws, by capillary attraction, the liquid from the reservoir into the capillary to bring the analyte and test reagent system into contact to facilitate reaction.

A variety of references describe small-volume fluid flow for a variety of purposes. U.S. Pat. No. 5,222,808 (Sugarman et al.), describes a capillary mixing device to allow mixing to occur in capillary spaces while avoiding the design constraints imposed by close-fitting, full-volume mixing bars. Mixing is facilitated by exposing magnetic or magnetically inducible particles, within the chamber, to a moving magnetic field.

U.S. Pat. No. 5,300,779 (Hillman et al.), describes a capillary flow device including a chamber, a capillary, and a reagent involved in a system for providing a detectable signal. The device typically calls for the use of capillary force to draw a sample into an internal chamber. A detectable result occurs in relation to the presence of an analyte in the system.

International patent publication no. WO 97/33737, published Mar. 15, 1996 by Kim et al., describes modification of surfaces via fluid flow through small channels, including capillary fluid flow. A variety of chemical, biochemical, and physical reactions and depositions are described.

While the above and other references describe useful techniques for chemical, biochemical and physical modification of surfaces, and analytical detection, a need exists for improved, small-scale fluid/surface interactions.

### SUMMARY OF THE INVENTION

The present invention provides a variety of techniques for altering a surface using fluid flow. Generally, the invention can involve creating adjacent components of a fluid stream exhibiting laminar flow, the first and second components mixing only via diffusion and being free of turbulent mixing. The first and second components are made to flow adjacent first and second portions, respectively, of a surface, and the first and second portions of the surface can be altered, selectively, using the components of the fluid stream. For example, at the first portion of the surface etching, plating, biological deposition, or the like can be carried out, while at the second portion of the surface a different situation can exist. At the second portion the same interaction can be effected as at the first portion, but to a different extent, or a different interaction or no interaction at all can be effected. For example, plating of a metal can occur at the first portion of the surface while nothing can occur at the second portion, or at the second portion of the surface plating to a different extent, or with a different metal, or etching can be made to occur. Alternatively, or in addition, fluid components can be selected where reaction between the fluid components, at the boundary therebetween, can effect a change at a surface against which the boundary flows. Etching, plating, or the like can be carried out in this manner alone, or can be carried out in addition to selective interactions effected by the first and second components of the fluid adjacent respective first and second portions of the surface. The surface can be tailored to be receptive to any of the described changes, selectively. The invention provides these techniques, and articles that can be produced by these techniques.

Other advantages, novel features, and objects of the invention will become apparent from the following detailed description of the invention when considered in conjunction with the accompanying drawings, which are schematic and which are not intended to be drawn to scale. In the figures, each identical or nearly identical component that is illustrated in various figures is represented by a single numeral. For purposes of clarity, not every component is labeled in every figure, nor is every component of each embodiment of the invention shown where illustration is not necessary to allow those of ordinary skill in the art to understand the invention.

### BRIEF DESCRIPTION OF THE DRAWINGS

FIGS. 1A, 1B, and 1C illustrate schematically, with photocopies of optical micrographs, laminar flow of multiple, converging fluid phases in a microfluidic system including demonstration of a luminescent chemical reaction at the boundary between two fluid phases (FIG. 1A), chemical etching adjacent one fluid phase selectively (FIG. 1B), and plating at the boundary between two fluid phases (FIG. 1C);

FIGS. 2A, 2B, 2C, and 2D illustrate schematically, with photocopies of optical micrographs, selective chemical reaction at a portion of a surface contacted selectively by a first component of a multi-component fluid stream, or at an interface of two components of a fluid stream;

FIGS. 3A, 3B, and 3C illustrate schematically, and via photocopies of scanning electron micrograph (SEM) images, chemical deposition at interfaces of components of flowing streams;

FIGS. 4A, 4B, 4C, 4D, and 4E illustrate schematically, and via photocopies of optical micrographs, stepwise fabrication of a three-electrode system inside a channel via selective chemical reaction within portions of the channel dictated by multi-component fluid streams;

FIGS. 5A and 5B illustrate schematically, and via photocopies of optical micrographs, patterns of adsorbed biological species dictated by multi-component fluid streams;

FIGS. 6A, 6B, 6C, 6D, and 6E illustrate schematically, and via photocopies of fluorescence micrographs (FIGS. 6A-6D) and a phase contrast image observed by an inverted microscope looking through a polystyrene Petri dish (FIG. 6E), deposition of biochemical agents, selectively, at portions of surfaces dictated by multi-component flowing streams;

FIGS. 7A, 7B, and 7C illustrate schematically, and via photocopies of phase contrast images, laminar flow used to etch channels to varying extents corresponding to components of a laminarly flowing stream;

FIGS. 8A, 8B, 8C, 8D, and 8E illustrates, schematically and via an SEM images and a CV spectrum, fabrication of an electronic device using laminar flow; and

FIG. 9 illustrates schematically, and via SEM image, laminar flow of gases to effect deposition.

### DETAILED DESCRIPTION OF THE INVENTION

The present invention provides techniques for carrying out chemical, biochemical, or physical reaction or deposition (hereinafter termed “interaction”) at surfaces from multi-component fluids generally via additive and/or subtractive processes. The interactions are carried out selectively at the surfaces in that they can occur proximate one component of the fluid to a greater extent than another component of the fluid, generally occurring proximate one component of the fluid to the exclusion of an area proximate a second component of the fluid. Alternatively, interactions can be made to occur selectively at interfaces of components of a multi-component fluid.

Components of a fluid can be arranged, relative to each other, via laminar flow. Techniques for facilitating laminar flow are known. Some known techniques involve creating side-by-side, parallel, contacting multiple flowing streams (or multiple components of a single stream) that are free of turbulent mixing, but are eventually allowed to mix by diffusion to allow analytical detection. The present invention utilizes laminar flow to create multi-component fluid streams that flow over an area of intended interaction with a surface, and are free of turbulent mixing across that area. Mixing occurs only via diffusion at interfaces of components of the fluid stream. While known techniques generally involve analytical detection, techniques of the invention generally involve altering a surface adjacent multi-component streams. Techniques of the present invention can be carried our by flowing fluid streams within channels of a variety of shapes and dimension. It is noted that the channels need not be straight, but can follow a non-linear path such as a curved path, zig-zag path, or other path.

A variety of techniques for creating laminar flow of fluid streams are known, and will not be discussed exhaustively in detail here. Those of ordinary skill in the art, upon reading the present disclosure, will be able to readily construct systems to carry out techniques of the invention.

The technique of the invention can be used to carry out essentially any additive or subtractive (adding material or removing material) chemical, biochemical, or physical reaction or deposition that can be promoted by a fluid proximate a surface, or between two fluids or two components of a fluid, optionally with the aid of electrical circuitry, magnetic fields, sonication, electromagnetic and other radiation, or the like. A variety of exemplary techniques suitable for use in creating interactions at surfaces in accordance with the invention include crystallization (precipitation); etching; electrochemical or electroless plating of metals, etc.; polymerization within a fluid proximate a surface, or at a fluid/surface interface, or at a fluid/fluid interface proximate a surface; biochemical interactions including specific biological binding interactions, adhesion of cells to surfaces, adsorption of species to surfaces, enzyme reactions, and the like. An exemplary, non-limiting list of species that can be patterned proximate a surface in accordance with the invention is described in International patent publication no. WO 97/33737, referenced above, and incorporated herein by reference. That is, the present invention is applicable to the patterning of metals, organic polymers, inorganic crystals, ceramics, and the like on any surface that supports laminar flow, typically on the inner walls of channels of any size that support laminar flow. These channels can be preformed capillaries. The preformed capillaries typically have diameters on the order of 20-100 microns, more often 50-100 microns. Because processes of the invention typically occur inside a pre-existing capillary, no registration steps are required.

Very small areas of interaction (reaction, deposition, etc.) at a surface, can be carried out using techniques of the invention. Species can be reacted or deposited at a surface in a pattern including a component or portion having a lateral dimension of less than 10 microns, preferably less than 5 microns, or less. These structures can be localized within a capillary with an accuracy of around 5 microns.

Techniques of the invention can be used in the application of structures as components of functional devices such as microelectrodes. In such systems, regions of a surface that may be desirably altered can range in length from tens of microns to several centimeters. The typical rate of flow of fluid streams of the invention in channels can be adjusted over a wide range, and typically is about 50 cm/s, and the time required for a volume element of fluid to traverse a length of 1 cm is 0.02 s. In these circumstances, turbulence is essentially eliminated, while diffusional mixing occurs over an interfacial layer of X_{diff} of around 6 microns.

Because many combinations of components of flowing streams can be generated by combining fluid streams using “Y” or “T” junctions (or their extensions to multiple streams), it is possible to bring a wide variety of solutions in contact with one another, and with the walls of a capillary, and to take advantage of the range of chemistries and other interactions available in these combinations to deposit material onto inner walls of the capillary, or to etch material from the inner walls of the capillary.

Related to biology and biochemistry, efforts to understand/the interaction of cells in culture with their environment can benefit from general procedures for patterning both the position of cells and the characteristics of the environment, that is, the molecular structure of the surface to which cells are attached, the nature and position of other cells in their vicinity, and the composition of a fluid medium surrounding them. Techniques of the present invention can be used to pattern substrate surfaces with adhesion promoters and inhibitors, to deliver cells to surfaces of a substrate in patterns, and to localize chemicals (e.g., fluorescent labels, nutrients, growth factors, toxins, enzymes, drugs, etc.) available to attached cells in the medium. Techniques of the invention enable new types of studies in fundamental cell biology and cellular metabolism, and are useful in the fabrication of analytical systems that use cells as sensors.

The ability to generate and sustain parallel streams of different solutions in capillaries provides the capability required to pattern (i) a substrate, by adsorption of adhesion promoters and inhibitors; (ii) the location of cells, by exposure of a pattern substrate to a suspension of cells, or by selective deposition of cells onto an unpatterned substrate from laminar streams; or (iii) a medium, by patterned flow. These methods easily generate patterns of parallel stripes and, using multi-step procedures described below, more complex patterns.

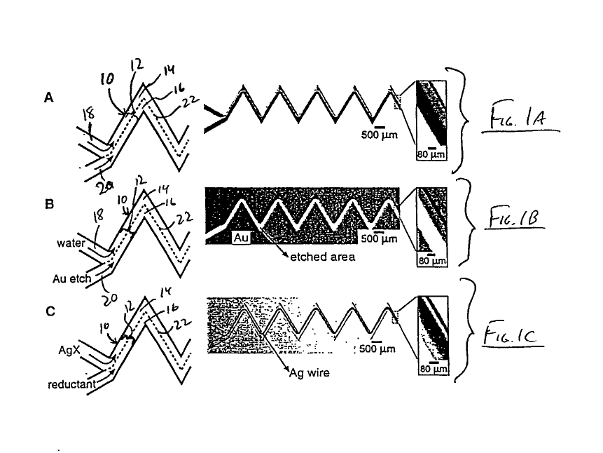

Referring now to FIGS. 1A-1C, one technique of the invention for carrying out fluid interactions with surfaces is illustrated. In each case, a channel 10 is provided that contains a fluid. Essentially any fluid or fluids can be used, including liquids and/or gases. The channel can be of any dimension or orientation in which laminar fluid flow can be promoted. The channel can be an enclosed capillary, a trough, or the like. In FIG. 1A, a flowing stream 12 of fluid is established within a channel defined by a polydimethylsiloxane (PDMS) membrane, with channels molded in its surface, sealed against the flat surface of a PDMS block. Such a system can be fabricated with reference to International patent publication WO 97/33737, referenced above. In such a system fluid flow can be controlled by surface tension, gravity, the application of electrical potentials, and/or both negative or positive pressure. (Pressure-driven flow was used in the examples of this application, below.) In stream 12, a first component 14 and a second component 16 are established within the channel, the first and second components supplied, respectively, by a first source 18 and a second source 20 defining separate conduits that join to form channel 10 at a “Y” junction. The dimensions of channel 10 are selected such that laminar flow maintains first and second components 14 and 16 in contact with each other, but free of turbulent mixing with each other.

Channels can be created in which one or more surfaces within the channel is preferentially amenable to chemical, biochemical, or physical reaction or deposition from fluid flowing within the channel. In such a case, where a boundary 22 between first and second components 14 and 16 of the fluid exists (at which diffusion can occur but turbulent mixing does not occur) where the boundary intersects the surface at which interaction is desired, the first component 14 can be provided with different potential for interaction with the surface, e.g., portions of the surface in contact with first component 14 can undergo interaction selectively (with no interaction occurring at portions adjacent fluid component 16) or preferentially, that is, to a degree greater than occurrence of the interaction at portions adjacent fluid component 16. First and second components 14 and 16 of the flowing stream are adjacent and parallel, and exhibit laminar flow.

The technique can be used to carry out chemical, biochemical, or physical interaction with a surface from component 14, but not fluid component 16, against a surface contacted by each where the surface is essentially uniform. That is, where a first portion of the surface contacted by fluid component 14 and a second portion of the surface contacted by fluid component 16 are essentially identical chemically and biochemically (neither having a preference for the interaction provided by the technique), chemical, biochemical, or physical interaction can be carried out, selectively, at one portion of a uniform surface but not at other portions, where the entire surface is contacted with a multi-component fluid stream. Another advantage of the invention is that a chemical, biochemical, or physical interaction can be effected at a surface where the interaction involves a fluid in a confined area of the surface, the interaction defining a lateral dimension less than that of the area of the confined fluid, i.e., in FIG. 1A, an interaction can occur involving first component 14, but not component 16, such that the results of the interaction exhibit a lateral dimension corresponding to component 14, where the entire confined fluid 12 has a larger lateral dimension.

In another set of embodiment, chemical, biochemical, or physical interaction is made to occur at boundary 22 between first and second components 14 and 16 of fluid stream 12, rather than at portions of a surface adjacent either of components 14 or 16, selectively. This can be done by selecting components 14 and 16 such that neither, alone, reacts with the surface at the intersection with boundary 22, but they react with each other to effect a chemical, biochemical or physical interaction. For example, two components 14 and 16 can be selected that, when mixed, plating or deposition of a metal occur, deposition occurring at the surface intersecting boundary 22 in a pattern corresponding to the boundary. This is another example, according to the invention, of carrying out a chemical interaction within a fluid-filled channel of a dimension significantly smaller than the channel itself. The surface of the channel contacted by the boundary is affected selectively, while other portions of the surface, within the channel, not contacted by the boundary are free of the interaction. Thus the invention allows for chemical, biochemical or physical interaction at a surface involving a fluid in a confined area of the surface, the interaction defining a lateral dimension of less than 50 microns, more preferably less than about 10 microns, more preferably less than about 5 microns. As mentioned, the lateral dimension is less than that of the area of the confined fluid.

Techniques of the invention result in articles including patterns having at least one edge corresponding to a fluid/fluid boundary. That is, the invention results in a substrate defining a surface including a first portion chemically or biochemically different from an adjacent second portion (the first portion defining the pattern created according to the technique of the invention). The first portion has a boundary that is the shape of a fluid/fluid boundary. The boundary of the pattern on the surface corresponds to the shape of boundary 22 as illustrated in FIG. 1A. A portion that “has a boundary that is the shape of a fluid/fluid boundary” is a portion that is fabricated from interaction at a fluid/fluid boundary.

One advantage of techniques of the invention is that patterns can be formed that do not necessarily correspond precisely to the flow path of a channel within which the patterns are formed. That is, “error correction” can be effected to correct surface irregularities within a channel. Where a chemical, biochemical, or physical interaction or deposition at a surface occurs at the intersection of the surface with a boundary between two components of a flowing stream, the boundary can be straight, or can change direction smoothly, where the interior surface of the channel within which the two fluid components flow is rough or otherwise not of a shape desired in a final pattern. Referring to FIG. 1C, it can be seen that a silver wire is deposited that zig-zags, changing direction smoothly rather than sharply as the channels change direction. In the system of FIG. 1C silver halide and a reductant are introduced into channel 10 as components 14 and 16, respectively, of fluid stream 12. Silver is deposited at boundary 22 between the components.

The invention involves establishing flowing streams of fluid against surfaces including first and second fluid components flowing adjacent first and second portions of a surface, respectively, and effecting a change at the first portion of the surface to a different, e.g., greater extent than at the second portion of the surface, or effecting a change at a portion of the surface adjacent a boundary between the first and second components of the fluid stream. The first portion of the surface can be changed in a predetermined way while the second portion of the surface is not changed at all, or the first portion of the surface can be changed in the same way as the second portion of the surface, but to a different extent, or the first and second portions of the surface can be changed in different ways, all with or without any change adjacent the boundary between the components. For example, the first portion of the surface can be plated with a metal, while the second portion of the surface can be plated with the same metal, but to a different extent, or can be plated with a different metal, or can be etched or treated with a biological agent, or can be left unchanged. At the same time, at the boundary between the two fluid components the surface can be etched, or plated, or the like, or left free of any change. Multiple fluid components can define multiple boundaries where each fluid component, and each boundary, can effect a different change adjacent the surface. Interactions that occur at a region of a surface to an extent different from another region of a surface can occur at least a 5 percent difference, 10 percent difference, 20, 30, 40 percent difference, or other percentage difference, typically in 10 percent increments, up to a 100 percent difference. A “percent difference”, in this context, means, for example, that if at a first portion of a surface X grams/cm² of a material is deposited, at a different region 0.9X grams/cm² may be deposited, defining a 10 percent difference. A 100 percent difference involves deposition of a material at one portion of a surface while a different portion of a surface remains free of deposition of the material.

It is also to be understood that portions of the surface adjacent components of fluid streams, or adjacent boundaries between fluid streams, can be altered to promote, specifically, a change at the surface. For example, a multi-component fluid stream can be created within a channel where reaction is promoted at one surface adjacent the boundary between the fluid channels because of pre-treatment at that surface, while another surface of the channel adjacent the opposite end of the boundary does not undergo alteration. Pre-treatment can involve coating with a self-assembled monolayer terminating in a chemical functionality that facilitates deposition, or the like.

It is another feature of the invention that alteration of a surface can be promoted by a flowing stream, where the portion of the surface altered is of a smaller dimension than that of the fluid stream. With reference to FIG. 1A-1C, it can be seen that fluid stream 12 can be established, while only a portion adjacent component 14 of the fluid stream can be altered, defining a portion of dimensions smaller than that of the fluid stream. Or a component of a surface adjacent boundary 22, significantly smaller than the dimension of fluid stream 12, can be altered.

The function and advantage of these and other embodiments of the present invention will be more fully understood from the examples below. The following examples are intended to illustrate the benefits of the present invention, but do not exemplify the full scope of the invention.

### EXAMPLE 1

### Microfluidic Laminar-Flow System

A system was assembled as illustrated in FIG. 1A from a PDMS block, to which was sealed a PDMS block having a zig-zag indentation to define a channel. An aqueous laminar flow fluid stream was established within the channel, defining two components. The first component (14 in FIG. 1A) contained Congo red dye and the second component (16) contained black ink. FIG. 1A shows a photocopy of an optical micrograph demonstrating good maintenance of unseparated, adjacent and parallel flowing streams 14 and 16 throughout the length of the channel 10 (dark portion is black ink, lighter portion is Congo red).

### EXAMPLE 2

### Selective Etching Adjacent One Component of Multi-Component Fluid Stream

Referring to FIG. 1B, selective etching of one portion of a surface in contact with a flowing stream is illustrated. In FIG. 1B, a PDMS article including an indentation defining channels (mold) was sealed against a glass slide covered with a thin (250 angstrom), semitransparent layer of gold. Parallel, laminar flow of water (first component 14) and an aqueous commercial gold etchant (second component 16) resulted in selective removal of gold at that portion of the surface in contact with second component 16 of the flowing stream. The photocopy of the optical micrograph of FIG. 1B shows channel 10 in which a section in contact with second component 16 is lighter, where gold was removed, but where an adjacent portion in contact with component 14 of the fluid stream maintains gold (darker portion).

### EXAMPLE 3

### Electroless Deposition at a Boundary of a Multi-Component Fluid Stream

Referring to FIG. 1C, in a system as described above with an PDMS article including an indentation in a flow pattern sealed against the flat surface of another PDMS block was used. Fluid stream 12 was established within channel 10 such that first and second components 14 and 16, respectively, defined two components of a commercial electroless silver plating solution. Specifically, component 14 included a silver halide and component 16 included a reductant. The result was deposition of silver at the boundary 22 of components 14 and 16, as can be seen in the photocopy of the resulting optical micrograph.

### EXAMPLE 4

### Deposition From One Component, Selectively, of Multi-Component Fluid Stream

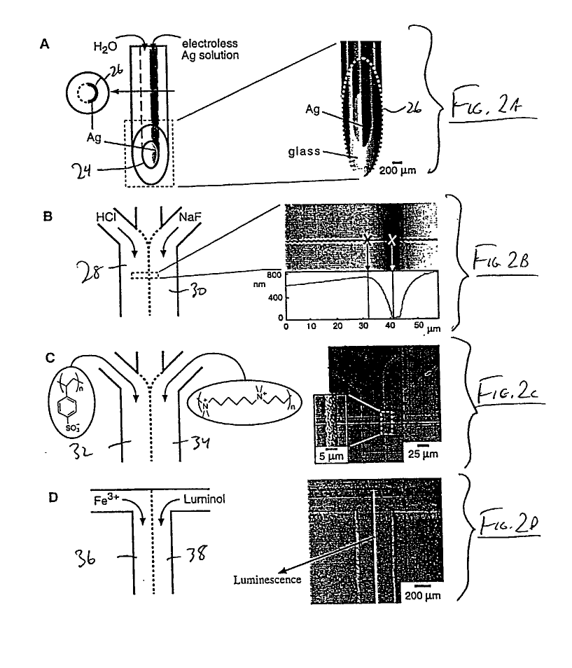

Referring to FIG. 2A, a glass capillary 24 was attached to a PDMS microfluidic “T” junction. Parallel laminar flow of water and of an aqueous phase containing the pre-mixed components of the silver plating solution described above deposited silver on one half of the inner surface of the glass capillary (section 26).

### EXAMPLE 5

### Selective Etching at Boundary of a Multi-Component Fluid Stream

Referring to FIG. 2B, a system as described above (with reference to FIG. 1B) was established as laminar components. First component 28 comprising HCl (2M in H₂O) and second component 30 comprising KF (2M in H₂O) were established. The result was a trench etched in the silicon oxide surface of a silicon wafer by the resulting HF generated at the boundary between the two fluid components. The half-width of the etched trench was 6 microns.

### EXAMPLE 6

### Precipitation of Polymeric Structure at Fluid/Fluid Boundary

A polymeric structure was precipitated at the interface of first and second components 32 and 34, respectively, of a flowing stream. Precipitation occurred at the boundary between two aqueous phases containing oppositely charged polymers flowing laminarly in parallel. First component 32 contained a 0.005% aqueous solution of poly(sodium 4-styrene-sulfonate) and second component 34 contained a 0.005% aqueous solution of hexadimethrine bromide.

### EXAMPLE 7

### Luminescence at Boundary of a Multi-Component Fluid Stream

Referring to FIG. 2D, a first component 36 and a second component 38 of a flowing stream within a channel were established where component 36 was K₃Fe_{III} (CN)₆ in water. Second phase 38 was a solution of luminol in 0.1 M aqueous NaOH. Luminescence at the boundary between components 36 and 38 resulted, as can be seen in the photocopy of the optical micrograph.

### EXAMPLE 8

### Deposition of Inorganic Crystals at Fluid/Fluid Boundary

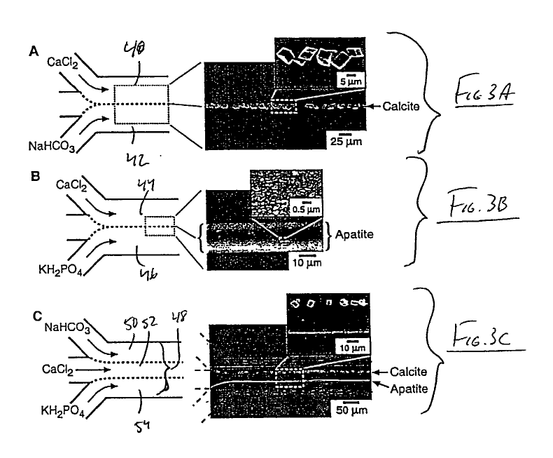

Referring to FIG. 3A, a two-component flowing stream was established in which a first component 40 was CaCl₂ (25 mM in H₂O) and component 42 was NaHCO₃ (100 mM in H₂O). Crystals were deposited on self-assembled monolayers of HS(CH₂)₁₅COOH on gold, where the SAM-coated surface was arranged to intersect the boundary of fluid components 40 and 42. Specifically, calcite single crystals were deposited as a result, as can be seen clearly in the accompanying photocopy of an SEM image.

Referring to FIG. 3B, a similar system involved a first component 44 similar to that of FIG. 3A but at half the concentration, and a fluid component 46 comprising KH₂PO₄ (3.6 mM in H₂O buffered to pH 7.4 with 0.1 M NaOH. Apatite was deposited as a result.

In FIG. 3C, a three-component fluid stream 48 is shown including first, second, and third components 50, 52, and 54, respectively. Component 50 was NaHCO₃ (16 mM in H₂O), pH 8.5). Component 52 was CaCl₂ (25 mM in H₂O) and component 54 was KH₂PO₄ (3.6 mM in H₂O, pH 7.4). The result was deposition, simultaneously, of calcite at a boundary between components 50 and 52 and apatite at a boundary between components 52 and 54.

### EXAMPLE 9

### Fabrication of Electronic Device Using Multi-Component Fluid Streams

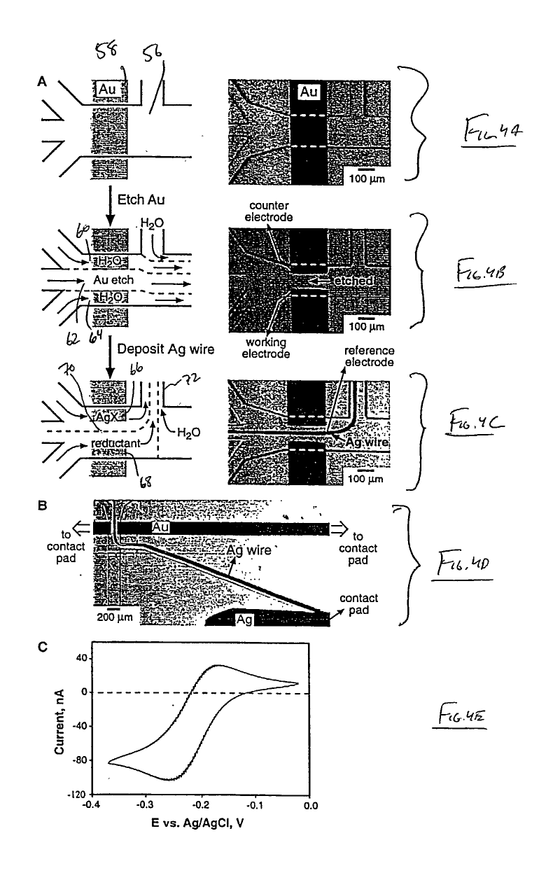

FIGS. 4A-4E show the stepwise fabrication of an array of three microelectrodes inside a rectangular capillary with a width of 200 microns (FIG. 4A). The capillary was assembled by placing a PDMS membrane that contained the channel network 56 on a flat surface of glass onto which a gold stripe 58 had been deposited by evaporation. Gold stripe 58 was oriented perpendicular to the main channel of channel network 56. A two-electrode system was generated by selectively removing gold by etching in the middle of the main channel of network 56 with a three-phase laminar flow system (FIG. 4B) of water (first component 60), gold etch (second component 62), and water (third component 64). The width of the etched area was controlled by controlling the relative volumes of the three liquid phases injected into the capillary, e.g. by varying pressure, surface tension, gravity, electrical potential, or the like. The remaining portions of the gold strip 58 defined two gold electrodes 66 and 68. A third, silver reference electrode was generated by depositing a silver wire at the inter-face of a two-component fluid stream generated in the channel. This boundary 70 of this two-component fluid stream fell between electrode 66 and 68, free of contact with either, using known silver plating fluid chemistry. Treatment with 1% HCl formed AgCl on the surface of the wire to form a Ag/AgCl reference electrode. The silver wire was directed into a small outlet 72 of the channel network using laminar flow. FIG. 4D shows an overview picture of the fabricated three-electrode system including a silver contact pad. The dashed box corresponds to the photocopy of the micrograph of FIG. 4C. FIG. 4E shows performance of the final device in cyclic voltammetry. Cyclic voltammetry was done in approximately 5 nL of Ru (NH₃)₆Cl₃ in water (2 mM with 0.1M NaCl as electrolyte) as recorded with the three-electrode system (100 mV/s) using the silver wire as the reference electrode and the two exposed gold areas as the counter and working electrode, respectively.

The technique described with respect to FIGS. 4A-4B illustrates one aspect of the invention that involves using fluid flow to create patterns on a surface that can be altered by altering the fluid flow. For example, in FIG. 4E fluid flow is promoted in a particular orientation, while in FIG. 4C changing fluid pressure in one channel of a multi-channel system causes fluid to flow in a different direction and via a different pattern with respect to several of the channels. This technique can be used to “write” one portion of an electrical circuit or the like along one channel, then a different portion along a different channel, etc., by altering fluid flow.

### EXAMPLE 10

### Patterning of Biological Species Using Multi-Component Fluid Flow

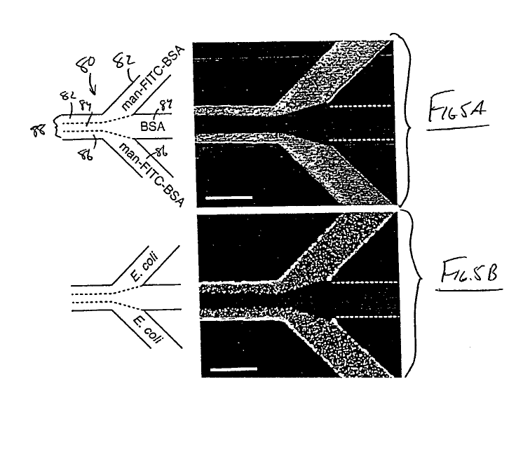

Referring now to FIGS. 5A and 5B, a capillary network 80 was fabricated by bringing a PDMS article having indentations corresponding to the capillary network into contact with the flat surface of a polystyrene petri dish. The capillary network included three inlet channels 82, 84, and 86 converging into a main channel 88. The capillary system was initially filled with water by filling the inlet reservoirs with water and applying vacuum at the outlet. Once liquid was flowing smoothly through the channels, a solution of a fluorescent neoglycoprotein, bovine serum albumin co-labeled with α-D-mannopyranosyl phenylisothiocyante and fluorescein isothiocynate (man-FITC-BSA) was placed in inlets 82 and 86, and a solution of BSA was placed in inlet 84. These solutions were allowed to flow into the main channel 88 forming corresponding components 82, 84, and 86 therein, under the influence of gentle aspiration at the outlet. Proteins adsorbed non-specifically to the regions of the surface over which the solutions containing them flowed. A photocopy of fluorescence microscopy visualized the resulting pattern (FIG. 5A). The system of capillaries was then filled with a suspension of E. coli RB 128, a strain that has been shown to bind to manose-presenting surfaces. Cells that did not adhere strongly were washed away with PBS. The remaining adherent cells were visualized with a fluorescent nucleic acid stain (FIG. 5B). E. Coli RB 128 adhered only to those portions of the channel that had been patterned by adsorption of man-FITC-BSA.

The petri dish was bacteriological, VWR. The man-FITC-BSA was from SIGMA 0.5 mg/mL, and the BSA was also SIGMA 10 mg/mL. After the man-FITC-BSA and BSA were allow to flow through the channel, the system was washed for 3 minutes with PBS (phosphate-buffered saline, pH 7.4). The E. coli RB 128 had been grown for 18 hours at 37□C in M9 media to NOD₆₀₀ of 1.2. After filling the channels with the E. coli suspension, they were allowed to stand for 10 minutes to allow adhesion. Cells were visualized with fluorescent nucleic acid stain (Syto 9, 15 microns in PBS, Molecular Probes). White dotted lines identify channels not visible with fluorescence microscopy. All scale bars are 100 microns.

### EXAMPLE 11

### Cell Patterns Via Multi-Component Fluid Streams

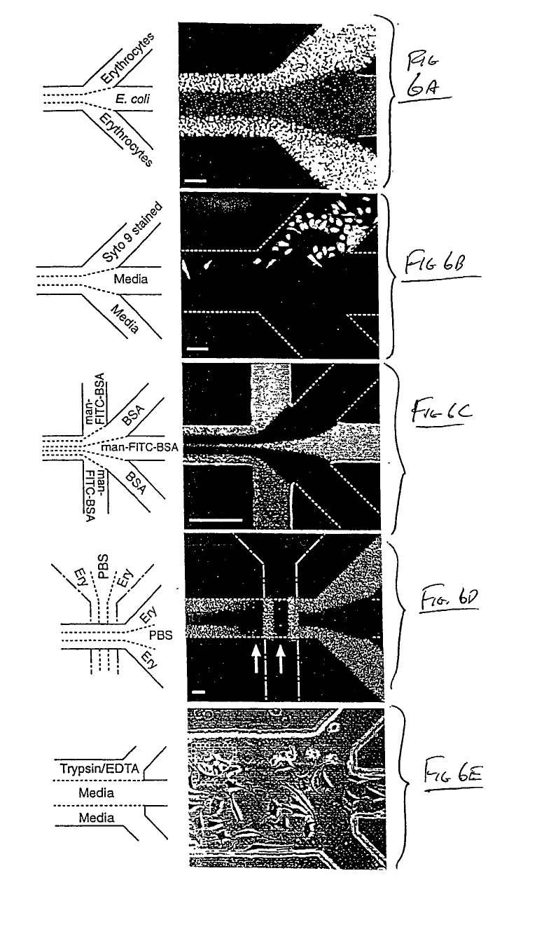

FIGS. 6A-6E illustrate various biological species patterns generated using laminar flow. FIG. 6A shows two different cells types patterned next to each other.

Specifically, a suspension of chick erythrocytes (12 day old, 5 mL cells in 165 mL Alsever's solution, SPAFAS Inc.) was placed in inlets 90 and 92, and PBS in inlet 2 and allowed to flow by gravitational force for 5 min followed by a 3 min PBS wash; this flow formed the pattern of bigger cells (outer lanes). Next, a suspension of E. coli (RB 128) was placed in inlet 94 and PBS in inlets 90 and 92 and allowed to flow by gravitational forces for 10 min followed by a 3 min PBS wash; this flow created the pattern of smaller cells (middle lane). Both cell types adhered to the Petri dish by non-specific adsorption. Cells were visualized with Syto 9 (15 μM in PBS). FIG. 6B: Pattern of stained bovine capillary endothelial (BCE) cells. BCE cells suspended in chemically defined medium (10 μg/mL high density lipoprotein, 5 μg/mL transferrin, 5 ng/mL basic fibroblast growth factor in 1% w/v BSA in Dulbecco's modified Eagle medium (DMEM), ˜10⁶ cells per mL)¹⁸ were introduced into the capillary network (pretreated with 50 μg/mL fibronectin for 1 hr) and incubated in 10% CO₂ at 37° C. for 4 hours. After removing non-adherent cells by washing with media (1% w/v BSA/DMEM, Gibco), Syto 9 (15 μM in BSA/DMEM) and media were allowed to flow from the designated inlets for 5 min under gentle aspiration, and the system was washed for 3 min with media. FIG. 6C: Pattern obtained at a junction where five inlets converge into a 75 μm channel. FIG. 6D: Criss-cross patterning of chick erythrocytes. Erythrocytes were patterned initially in the vertical direction. The PDMS membrane used in this initial pattering was demounted and a different PDMS membrane placed in a direction 90° rotated from the first pattern (that is, horizontal). The capillary system created by the second PDMS membrane was used to pattern erythrocytes in the horizontal (right to left) direction. The resulting pattern of erythrocytes in the second capillary system was visualized by Syto 9 (15 μM in PBS). FIG. 6E: Patterned detachment of BCE cells by treatment with trypsin/EDTA. Cells were allowed to adhere and spread in a fibronectin treated capillary network for 6 hrs, and non-adherent cells removed by washing. Trypsin/EDTA (0.05% trypsin/0.53 mM EDTA, Gibco) and media were allowed to flow from the designated inlets for 12 min by gravity. White dotted lines identify channels not visible with fluorescence microscopy. All scale bars, 100 μm.

### EXAMPLE 12

### Selective Etching of PDMS

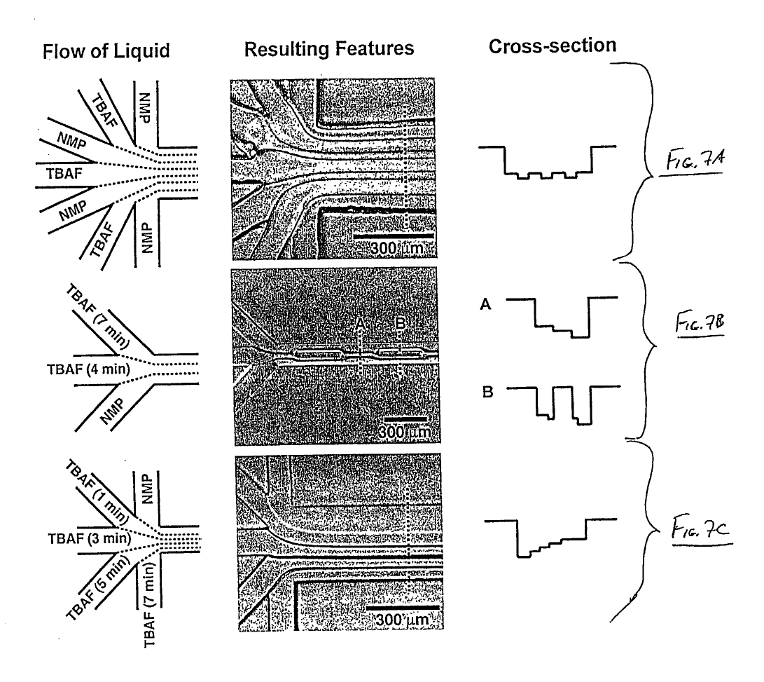

Referring to FIGS. 7A-7C, multi-component fluid streams selectively dissolves PDMS to create channels of varying profiles or cross-sections. FIG. 7A shows alternating NMP and TBAF component fluid streams. Because TBAF selectively dissolves PDMS, parallel laminar flow of TBAF on a PDMS channel controllably removes PDMS within the channel creating steps or secondary level structure in the PDMS. The cross-sectional profile is shown. FIG. 7B shows that the time-controlled flow of parallel streams of TBAF in PDMS may be used to create varying cross-sectional profiles within a flow channel. A two-component flow of NMP with TBAF was later changed to a three-component flow to create the cross-sectional profile shown. FIG. 7C shows a time-wise varied multi-component flow of TBAF in PDMS to create the progressively deepening removal of PDMS by TBAF.

### EXAMPLE 13

### Fabrication of Electronic Devices Using Multi-Component Fluid Streams

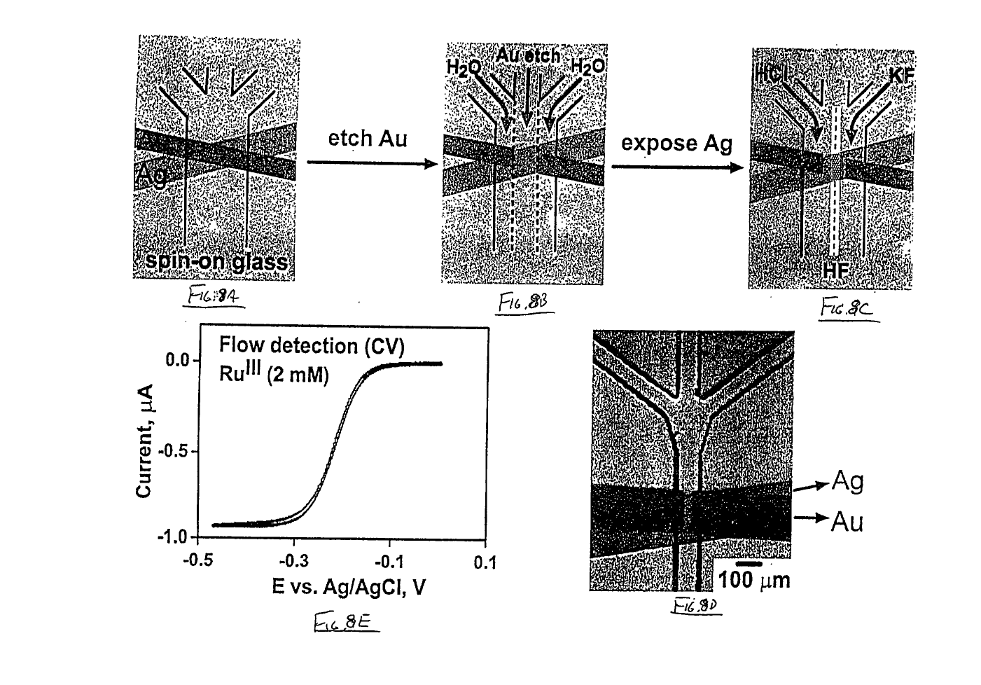

FIG. 8A-8E show the stepwise fabrication of a three microelectrode electrochemical detector with a width of less than 100 microns. A silver electrode was deposited on the surface substantially perpendicular to the channel. Spin-on glass was used to isolate the silver electrode. The gold electrode was deposited on the spin-on glass layer. (FIG. 8A). The electrode was created by selectively removing gold in the middle of the channel with a three-component laminar flow system of water, gold-etch and water to expose a portion of the spin-on glass layer. (FIG. 8B). The spin-on glass layer was then selectively etched with a three-component laminar flow system of HCl, HF, and KF to expose the silver layer. (FIG. 8C). The detector thus created is shown in FIG. 8D. FIG. 8E shows the performance of the device in cyclic voltammetry under similar conditions as Example 9 except that flowing electrolyte was used.

### EXAMPLE 14

### Gaseous Laminar Flow

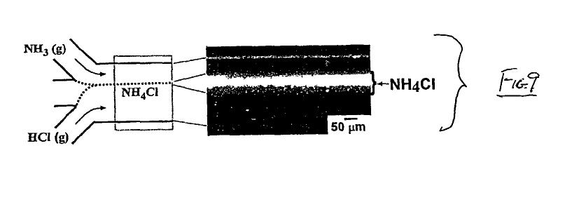

FIG. 9 illustrates schematically, and via an SEM image, use of laminar flow of gaseous components of a fluid stream to effect deposition and a boundary between the components. Dry gaseous NH₃ (approximately 10 percent in air) as components 100 and dry gaseous HCl (approximately 10 percent in air) as components 102 were flown laminarly in a 400 micron-wide PDMS channel 104. White solid NH₄Cl formed at the interface between components 100 and 102.

Those skilled in the art would readily appreciate that all parameters listed herein are meant to be exemplary and that actual parameters will depend upon the specific application for which the methods and apparatus of the present invention are used. It is, therefore, to be understood that the foregoing embodiments are presented by way of example only and that, within the scope of the appended claims and equivalents thereto, the invention may be practiced otherwise than as specifically described. In the claims the words “including”, “carrying”, “having”, and the like mean, as “comprising”, including but not limited to.

What is claimed is:

1. A method comprising:
establishing a flowing stream of a fluid against a surface, the stream including first and second components in contact with first and second portions of the surface, respectively, the first component carrying different potential for a chemical, biochemical, or physical interaction than the second component; and
carrying out the chemical, biochemical, or physical interaction at the first portion of the surface to an extent different than at the second portion of the surface.

2. A method as in claim 1, wherein the first and second components of the fluid stream differ in that the first component carries the potential for the chemical, biochemical, or physical interaction and the second component does not.

3. A method as in claim 1, wherein the first and second components of the fluid stream are adjacent and parallel.

4. A method as in claim 1, wherein the first and second components of the fluid stream are adjacent and parallel portions exhibiting laminar flow.

5. A method comprising:
establishing a flowing stream of a fluid against a surface, the stream including first and second components in contact with first and second portions of the surface, respectively, the first and second portions of the surface being essentially identical chemically and biochemically; and
carrying out a chemical, biochemical, or physical interaction at the first portion of the surface to an extent different than at the second portion of the surface.

6. A method comprising:
establishing first and second adjacent and parallel flowing streams of fluid against a surface, the first and second streams in contact with first and second portions of the surface, respectively, the first and second portions of the surface being essentially identical chemically and biochemically; and
carrying out a chemical, biochemical, or physical interaction at the first portion of the surface to an extent different than at the second portion of the surface.

7. A method as in claim 6, involving carrying out the chemical, biochemical, or physical interaction at the first portion of the surface while leaving the second portion of the surface free of the interaction.

8. A method as in claim 6, further comprising carrying out a second chemical, biochemical, or physical interaction at the second portion of the surface.

9. A method comprising:
carrying out a chemical, biochemical, or physical interaction at a surface involving a fluid in a confined area of the surface, the interaction having a lateral dimension less than that of the area of the confined fluid.

10. A method comprising:
establishing a flowing stream of a fluid, the stream including first and second components in contact with each other and defining therebetween a boundary;
carrying out a chemical, biochemical, or physical interaction at a first portion of a surface of a substrate proximate the boundary selectively, to an extent different than at the second portion of the surface.

11. A method as in claim 10, comprising carrying out a second chemical, biochemical, or physical interaction at the second portion of the surface.

12. A method as in claim 10, further comprising leaving a second portion of the surface free of the chemical, biochemical, or physical interaction.

13. A substrate defining a surface including a first portion chemically or biochemically different from an adjacent second portion, the first portion having a boundary of the shape of a fluid/fluid boundary.

14. A substrate defining a surface including a first portion chemically or biochemically different from an adjacent second portion caused by chemical, biochemical, or physical interaction involving a fluid at the first portion, the first portion having a having a lateral dimension of less than 1 micron.

15. A method as in claim 1, wherein the flowing stream includes at least a third component in contact with at least a third portion of the surface.

16. A method as in claim 15 involving carrying out the chemical, biochemical, or physical interaction of the third component for a duration equal to or less than the duration for the first or second components.

17. A method as in claim 6, wherein at the flowing stream includes at least a third component in contact with at least a third portion of the surface.

18. A device including a first electrode in electrical isolation from a second electrode and at least a third electrode also in electrical isolation from the first and second electrodes having a potential for a chemical, biochemical, or physical interaction with a fluid.

19. A method comprising:
providing a first electrically-conductive material, an electrically non-conductive material on the first electrically-conducted material, and a second electrically-conductive material adjacent the electrically non-conductive material;
via laminar flow of at least two components of a fluid stream, removing a portion of the second electrically-conductive material thereby exposing a portion of the electrically nonconductive material; and
via laminar flow involving at least two components of a fluid stream, removing a portion of the electrically non-conductive material thereby exposing at least a portion of the first electrically-conductive material, and establishing thereby exposed portions of the first and second electrically-conductive materials separated by the electrically non-conductive material.
